(19) United States
(12) Patent Application Publication
(10) Pub. No.: US 2005/0209672 A1
(43) Pub. Date: Sep. 22, 2005

(54) Sliding restraint stent delivery systems

(75) Inventors: William R. George, (US); Jean C. Chang, Saratoga, CA (US); Dai T. Ton, Milpitas, CA (US); Nicholas C. De Beer, Montara, CA (US); Julian Nikolchev, Portola Valley, CA (US); Frank Becking, Palo Alto, CA (US)

Correspondence Address: BOZICEVIC, FIELD & FRANCIS LLP (CARDIOMIND), 1900 UNIVERSITY AVENUE, SUITE 200, EAST PALO ALTO, CA, 94303, US

(73) Assignee: CardioMind, Inc.

(21) Appl. No.: 10/792,684

(22) Filed: Mar. 2, 2004

(51) Int. Cl. A61F 2/06

(52) U.S. Cl. 623/1.11

(57) ABSTRACT

Medical device and methods for delivery or implantation of prostheses within hollow body organs and vessels or other luminal anatomy are disclosed. The subject technologies may be used in the treatment of atherosclerosis in stenting procedures. For such purposes, a self-expanding stent may be deployed in connection with an angioplasty procedure with a sliding restraint based delivery system adapted for simplified use. In the system, the sliding restraint is sized, in coordination with a fixed sleeve accepting a core wire to actuate the restraint to effect an anchoring function with the sleeve so that the stent is not inadvertently advanced during deployment.

### FIELD OF THE INVENTION

The present invention relates generally to medical devices and methods. More particularly, it relates to delivery systems for implanting prostheses within hollow body organs and vessels or other luminal anatomy.

### BACKGROUND OF THE INVENTION

Implants such as stents and occlusive coils have been used in patients for a wide variety of reasons. One of the most common “stenting” procedures is carried out in connection with the treatment of atherosclerosis, a disease which result in a narrowing and stenosis of body lumens, such as the coronary arteries. At the site of the narrowing (i.e., the site of a lesion) a balloon is typically dilatated in an angioplasty procedure to open the vessel. A stent is set in apposition to the interior surface of the lumen in order to help maintain an open passageway. This result may be effected by means of scaffolding support alone or by virtue of the presence of one or more drugs carried by the stent aiding in the prevention of restenosis.

Various stent designs have been developed and used clinically, but self-expandable and balloon-expandable stent systems and their related deployment techniques are now predominant. Examples of self-expandable stents currently in use are the Magic WALLSTENT® stents and Radius stents (Boston Scientific). A commonly used balloon-expandable stent is the Cypher® stent (Cordis Corporation). Additional self-expanding stent background is presented in: “An Overview of Superelastic Stent Design,” Min. Invas Ther & Allied Technol 2002: 9(3/4) 235-246, “A Survey of Stent Designs,” Min. Invas Ther & Allied Technol 2002: 11(4) 137-147, and “Coronary Artery Stents: Design and Biologic Considerations,” Cardiology Special Edition, 2003: 9(2) 9-14, “Clinical and Angiographic Efficacy of a Self-Expanding Stent” Am Heart J 2003: 145(5) 868-874.

Because self-expanding prosthetic devices need not be set over a balloon (as with balloon-expandable designs), self-expanding stent delivery systems can be designed to a relatively smaller outer diameter than their balloon-expandable counterparts. As such, self-expanding stents may be better suited to reach the smallest vasculature or achieve access in more difficult cases.

To realize such benefits, however, there continues to be a need in developing improved delivery systems. Problems encountered with known systems include drawbacks ranging from failure to provide means to enable precise placement of the subject prosthetic, to a lack of space efficiency in delivery system design. Poor placement hampers stent efficacy. Space inefficiency in system design prohibits scaling the systems to sizes as small as necessary to enable difficult access or small-vessel procedures (i.e., in tortuous vasculature or vessels having a diameter less than 3 mm, even less than 2mm).

One known stent delivery system comprises a simple sheath set over a pusher in abutment with a stent. An example of such a system is disclosed in U.S. Pat. No. 4,580,568. Though elegant in design, the system fails to offer desired functional characteristics. Particularly, such a system is prone to misuse when a physician who is not intimately familiar with the hardware retracts or pushes the wrong one of the stent-abutting member or the sheath in an effort to free the stent. Dedicated handle systems have been developed to address this problem. Examples are provide in WO 99/04728, WO 00/18330, WO 98/23241, EP-A-747021, DE-A-44 20142 and U.S. Pat. No. 5,433,723.

Even when not misused, simple sheath systems present issues with precise stent placement stemming from the fact that the sheath cannot be locked-down at the proximal end of an access catheter (e.g., at a hemostatic valve) while deploying the stent. As a result, it is difficult to prevent inadvertent axial movement of the stent. Because the sheath cannot be held stationary, stent deployment requires that a user hold the pusher member (or handle attached thereto) steady while withdrawing the sheath in order to avoid pushing the stent forward within the vessel. Such forward movement complicates stent placement and ma cause vessel injury due to the leading edges of the stent scraping against (producing “skid marks”), or even perforating the vessel wall.

However, careful actuation of a sheath-based delivery system will not ensure precise stent placement. It is often further required that the user draw any appreciable slack out of the system before attempting to withdraw the sheath. If not, slack in the delivery system either outside or inside of the patient can result in deploying the stent beyond the target site. While counterintuitive, this result occurs because of a balance of number of factors detailed below.

In fact, this observed behavior is not unique to simple-sheath stent delivery system designs. Even though the system described in U.S. Pat. No. 5,534,007 assigned to SciMed Life Systems, Inc. offers an alternative to a simple-sheath type system for deploying self-expandable stents in which the hemostatic valve at a proximal end of a delivery catheter receiving the device can be tightened down onto the same (thus avoiding unintended axial movement during stent positioning), the instructions for use of the system still direct drawing the slack out of the system. Yet, depending on the nature of the target site environment (i.e., the size and tortuosity of the subject anatomy), testing of the Radius™ system covered by the '007 patent by the inventors hereof has demonstrated the same forward-wandering tip/stent movement produced in a simple sheath system.

While FIGS. 4-7 of the '007 patent do disclose an alternative embodiment not prone to the forward stent/tip wandering as described above, the embodiment is not believed amenable to or adaptable for use in reaching very small, largely occluded or highly tortuous vasculature of the type described above. The reason for this statement is two-fold. First, the device includes a guidewire lumen. That is, the device is a standard “over-the-wire” type delivery device. This fact, alone, requires that the delivery device cross-sectional diameter be appreciably larger than that of a common guidewire (at minimum, it includes several layers overriding the guidewire). Second, a bellows-type retractable restraint is set directly adjacent the distal end of the device. Even from the patent figures, it is clear that such a structure must grow appreciably in diameter from its no-fold profile to fully-folded profile.

To remedy one or more of the shortfalls noted above, it would be desirable to produce a delivery system having a cross-section at or about that of a common guidewire size between about 0.010 inch (0.25 mm) and 0.035 inch (0.89 mm) that is configured to avoid unintended (often, forward) tip/stent movement during deployment of the same. Those with skill in the art may also appreciate further advantages or benefits potentially offered by the present invention.

### SUMMARY OF THE INVENTION

The present invention offers a high-precision stent placement delivery system, allowing a user to conveniently “lock-down” the system, if desired, and deliver a stent thus set in place. The system includes a stent and a delivery guide for carrying the stent to a treatment site and releasing the stent at that point. To facilitate the lock-down function, the delivery guide is configured such that it is actuated by a member interior to an outer sleeve onto which the hemostatic valve attached or connected to a catheter (e.g., a microcatheter or balloon catheter) can be collapsed. The inner member may be a core member (i.e., filling the center of or being coaxial with the sleeve) or one of a number of inner members.

The delivery guide further employs a distal restraint that holds the stent in a collapsed configuration in an undeployed state at a distal end portion of the delivery device. Actuation of the interior member (e.g., by withdrawing the same or by a physical shortening, such as by a heat-activated shape memory plastic or alloy wire) at a proximal end of the delivery system deploys the stent by way of withdrawal or retraction of the distal restraint from about the stent. In doing so, the restraint holding the stent or a connection thereto may pass from the outside of the delivery device to inside its body. Such an approach facilitates a low-profile delivery device that does not change in diameter during use.

In certain variations of the invention, a connector segment is provided as an attachment between the inner member and the restraint. The connector may be substantially or entirely exposed, or have a portion that underlies a section of the outer sleeve. The connector passes from the outside of the device into the sleeve, along with the inner/core member or wire used to actuate the system. The restraint may be a simple tubular member or a plurality of members working in concert. A full description of exemplary sliding restraint stent delivery systems is presented in Attorney Docket No. CRMD-005, entitled “Corewire Actuated Delivery Systems with Fixed Distal Stent-Carrying Extension” filed on even date herewith and incorporated by reference herein in its entirety.

Alternatively, the restraint employed in the present invention may be adapted to assume a reduced diameter upon withdrawal (itself) into the sleeve. A full discussion of exemplary diameter adaptive restraint technology systems is provided in Atty. Docket No. CRMD-006, entitled, “Stent Delivery System with Diameter Adaptive Restraint”, also filed on even date herewith and also incorporated by reference herein in its entirety.

Irrespective of such (independently inventive) details, a preferred variation of the invention includes an end configuration of the device 1) that does not increase in size from its loaded, stent-carrying state to its post-deployment state, and 2) that allows the restraint to move freely relative to opposing anatomy in moving off of the stent it restrains to effect stent release. Other preferred variations of the invention will include only one or the other of these two characteristics. However, all of the variations of the invention involve stent deployment by actuation of a member interior to an outer member.

The subject delivery systems, and more particularly the delivery guide, preferably includes an atraumatic tip distal to the restraint. The system may also comprise a user-friendly handle. The stent itself will generally be self-expanding upon release from the restraint. Thus, full or complete placement of the stent is achieved solely upon its release from the delivery device. Still, aspects of the invention may be applicable to balloon-expandable stent delivery systems.

Delivery systems and guides according to the present invention are amenable to scaling to sizes not previously achieved. Consequently, the systems may be used in lieu of a guidewire, such as in a “guidewireless” delivery approach. Still further, rather than providing an “over-the-wire” delivery system as referenced above, the present systems may be regarded as “on-the-wire” delivery systems, since—in effect—delivery is accomplished by a system in which the stent is carried by a delivery guide occupying a catheter lumen that would commonly otherwise be used to accommodate a guidewire.

Whether used in such a manner or otherwise (such as by configuring the subject systems for treating larger peripheral vessels), the present invention includes systems comprising any combination of the features described herein. Methodology described in association with the devices disclosed also forms part of the invention. Such methodology may include that associated with completing an angioplasty, bridging an aneurysm, deploying radially-expandable anchors for pacing leads or an embolic filter, or placement of a prosthesis within neurovasculature, an organ selected from the kidney and liver, within reproductive anatomy such as selected vasdeferens and fallopian tubes or other applications.

Where the systems are adapted for use in small and/or tortuous anatomy, each of the above-referenced optional performance characteristics of the device offers great potential benefit. In the first instance, the adaptation so the distal end of the device does not increase in its overall diameter from pre-deployment to post-deployment allows for the use of a closely-sized delivery catheter. An example of such a small delivery lumen is that a balloon catheter—one such means of delivery as advantageously employed in connection with the present invention. Details of such a mode of deploymement is presented in U.S. Patent Application Atty. Docket No. CRMD-003, entitled “Balloon Catheter Lumen Based Stent Delivery Systems,” filed Dec. 24, 2003 and incorporated herein by reference in its entirety. Also, by avoiding delivery device distal diameter increase risk of embolizing vulnerable plaque may be decreased. Still further, the substantially constant system distal diameter helps avoid unintended additional vessel occlusion during a procedure.

The subject delivery systems may be further or alternatively adapted to effect precise stent placement by avoidance of unintended forward stent movement upon actuation. Precise placement of a stent can becritical due to the need to avoid occlusion of a sidebranch, overlapping an adjacent stent or ensuring proper stent/vessel sizing matchup. Therefore, the potential benefit of placement with motion is highly desired.

To help ensure proper stent placement, the delivery guide may be adapted so that the section holding or abutting the stent will not move forward (axially).when withdrawing the restraint from about the stent. In some cases, this will mean that the distal tip of the device and/or the stop abutting the stent does not move upon restraint withdrawal.

However, systems are contemplated in which an atraumatic tip is provided that is retracted along with the restraint. In any case, a stop or blocker is provided to maintain the stent (whether it be a raised shoulder on a core member, an end of the sleeve or otherwise provided) in a stationary position relative to the delivery system body upon restraint withdrawal.

Such action is provided by virtue of coordination of any of a number of features. One such feature is the length of the restraint. A longer restraint will be more prone to gripping opposing tortuous anatomy as there is more surface area for friction to act upon. A shorter restraint will not suffer from significant stiction (static friction breakaway force) in this manner. The specific length of the restraint will depend on the subject anatomy the device is intended to operate within. Exemplary lengths are provided below for coronary anatomy applications.

In addition to the length of the restraint, where connector portions are provided that are not covered by the sleeve, their configuration may be important as well. Still further, stiction of the restraint may be controlled for the restraint by utilizing lubricious material in its production or by providing a lubricious coating.

Also of importance are the physical parameters of the sleeve, especially those portions of the sleeve where adjacent to the restraint and/or connectors. In this aspect of the invention, it is intended that the sleeve be of such a length that it be introducible into the tortuous anatomy along with the restraint.

Unlike the restraint, however, the sleeve (at least in this section) may be adapted to avoid movement relative to the anatomy to be treated. This character may result simply from the length disposed within the tortuous anatomy, causing certain binding points or friction therewith, or by coatings, a lack of a lubricious coating and/or surface finish.

However accomplished, the systems are adapted to allow for precise stent placement without unintended forward movement of the delivery device portion carrying the stent. Depending on the subject anatomy into which it is introduced, a selected system having one length of restraint and/or connectors (or having the sleeve set back a certain distance from the distal tip) will result in the functional characteristics desired while another similarly constructed, but differently-sized system will not.

Accordingly, an aspect of the present invention is in the provision of a number of differently-sized systems in a kit, or a panel (as in an optical setting), or other common storage location from which to select to achieve the performance described. The systems may be sized in terms of properly effecting stent placement relative to relevant anatomy (e.g., as in having a system sized with a distal end of the sleeve (or an between about 25 cm and about 30 cm, or about 20 cm or about 25 cm, or about 10 cm and about 15 cm distal to the ostium when in use).

At a minimum, the restraint will typically be at least about as long as the stent or stents it is to release. However, an exposed length of restraint and/or any connection thereto may be between about a length of the stent and about 5 cm long, between about 5 cm and about 10 cm long, or about 10 cm and about 20 cm long and still be of use in according to the subject invention in coronary procedures. For use in other settings, such as in effecting neurovasculature, renal or liver/hepatic treatments, the length may be different. However, the same systems might be used (selected from).

As such, there are other ways (as opposed to strict dimensions) to characterize the required structural characteristic of the delivery device. Specifically, it can be appreciated in functional terms in reference to the anatomy in which the device is to perform as intended. In this regard, the anatomy may be a generic body conduit that is tortuous in nature sufficiently that it will bind other devices to result in unwanted tip/stent movement, instead of intended precise location. Relevant subject locations include sites within the microvasculature, kidney, liver, another body organ or the reproductive anatomy including the vasculature, vasdeferens and fallopian tubes.

However described, the selection of devices have restraints configured to be so-bound by adjacent anatomical structure, even with appreciable slack in the system, a to prevent axial movement of the stent while in the process of releasing it. Specifically such tortuous anatomy may have a diameter of less than about 3 mm over a length of at least about 5 cm, the diameter may be smaller—as in about 2.5 mm, to 2.0 mm or less. In addition, the section of tortuous anatomy to be traversed by the delivery device or guide to reach a treatment site may be longer as in about 10 cm, 15 cm or more at the stated diameters.

### DEFINITIONS

The term “stent” as used herein refers to any coronary artery stent, other vascular prosthesis, or other radially expanding or expandable prosthesis or scaffold-type implant suitable for the noted treatments or otherwise. Exemplary structures include wire mesh or lattice patterns and coils, though others may be employed in the present invention.

A “self expanding” stent is a scaffold-type structure (serving any of a number of purposes) that expands by its own action from a reduced-diameter configuration to an increased-diameter configuration. The “diameter” need not be circular—it may be of any open configuration. Self-expanding materials may be so by virtue of simple elastic behavior, superelastic behavior, a shape memory effect (i.e., heat-activated transformation from martinsite to austenite) or some other manner. Since the stents will remain in the subject's body, the material should be biocompatible or at least be amenable to biocompatible coating. As such, suitable self expanding stent materials for use in the subject invention include Nickel-Titanium (i.e., NiTi) alloy (e.g., NITINOL) and various other alloys or polymers.

A “wire” as used herein generally comprises a common metallic member. However, the wire may be coated or covered by a polymeric material (e.g., with a lubricious material such as TEFLON®) or otherwise. Still further, the “wire” may be a hybrid structure with metal and a polymeric material (e.g. Vectra™, Spectra™, Nylon, etc.) or composite material (e.g., carbon fiber in a polymer matrix). The wire may be a filament, bundle of filaments, cable, ribbon or in some other form. It is generally not hollow.

A “core” wire as referred to herein is a member internal to an outer member, such as a tubular member. As a core wire, the member, fills or at least substantially fills all of the interior space of the tubular member.

A “hypotube” or “hypotubing” as referred to herein means small diameter tubing in the size range discussed below, generally with a thin wall. The hypotube may specifically be hypodermic needle tubing. Alternatively, it may be wound or braided cable tubing, such as provided by Asahi Intec Co., Ltd or otherwise. As with the “wire” discussed above, the material defining the hypotube may be metallic, polymeric or a hybrid of metallic and polymeric or composite material.

A “sleeve” as referred to herein may be made of such hypotubing or otherwise. The sleeve may be a tubular member, or it may have longitudinal opening(s). It is an outer member, able to slidingly receive and hold at least a portion of an inner member.

An “atraumatic tip” may comprise a plurality of spring coils attached to a tapered wire section. At a distal end the coils typically terminate with a bulb or ball that is often made of solder. In such a construction, the coils and/or solder is often platinum alloy or another radiopaque material. The coils may also be platinum, or be of another material. In the present invention, the wire section to which the coils are attached may be tapered, but need not be tapered. In addition, alternate structures are possible. For instance, molding or dip-coating with a polymer may be employed. In one example, the atraumatic tip may comprise a molded tantalum-loaded 35 durometer Pebax™ tip. However constructed, the atraumatic tip may be straight or curved, the latter configuration possibly assisting in directing or steering the delivery guide to a desired intravascular location.

To “connect” or to have or make a “connection” between parts refers to fusing, bonding, welding (by resistance, laser, chemically, ultrasonically, etc), gluing, pinning, crimping, clamping or otherwise mechanically or physically joining, attaching or holding components together (permanently or temporarily).

### BRIEF DESCRIPTION OF THE DRAWINGS

Each of the figures diagrammatically illustrates aspects of the invention. Of these:

FIG. 1 shows a heart in which its vessels may be the subject of one or more angioplasty and stenting procedures;

FIG. 2 shows an expanded stent cut pattern as may be used in producing a stent for use in the present invention;

FIGS. 3A-3L illustrate stent deployment methodology to be carried out with the subject delivery guide member;

FIGS. 4A-4C illustrate the stent deployment activity commented upon in the Background section above. FIGS. 4A and 4B explain observed forward-wandering stent deployment behavior in a simple-sheath based delivery system; FIG. 4C illustrates the proper mode of deploying a stent with such a known system.

FIGS. 5A and 5B illustrate the manners in which stent deployment may occur with an inner member actuated system;

FIG. 6A is an enlarged view of the relation between a diagramatic section of tortuous vasculature and a simple sheath delivery system; FIG. 6B shows another delivery system in the same diagrammatic section of vasculature that suffers the same effect as the simple-sheath system; FIG. 6C shows a delivery system according to the present invention positioned within the same diagrammatic section of vasculature;

FIG. 7 shows an overview of a delivery system according to the present invention and its associated packaging; and

FIGS. 8A-8C provide detailed views of a first restraint approach applicable the present invention; FIG. 9 details a second restraint approach applicable to the present invention.

Variation of the invention from the embodiments pictured is, of course, contemplated.

### DETAILED DESCRIPTION OF THE INVENTION

Before the present invention is described in detail, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

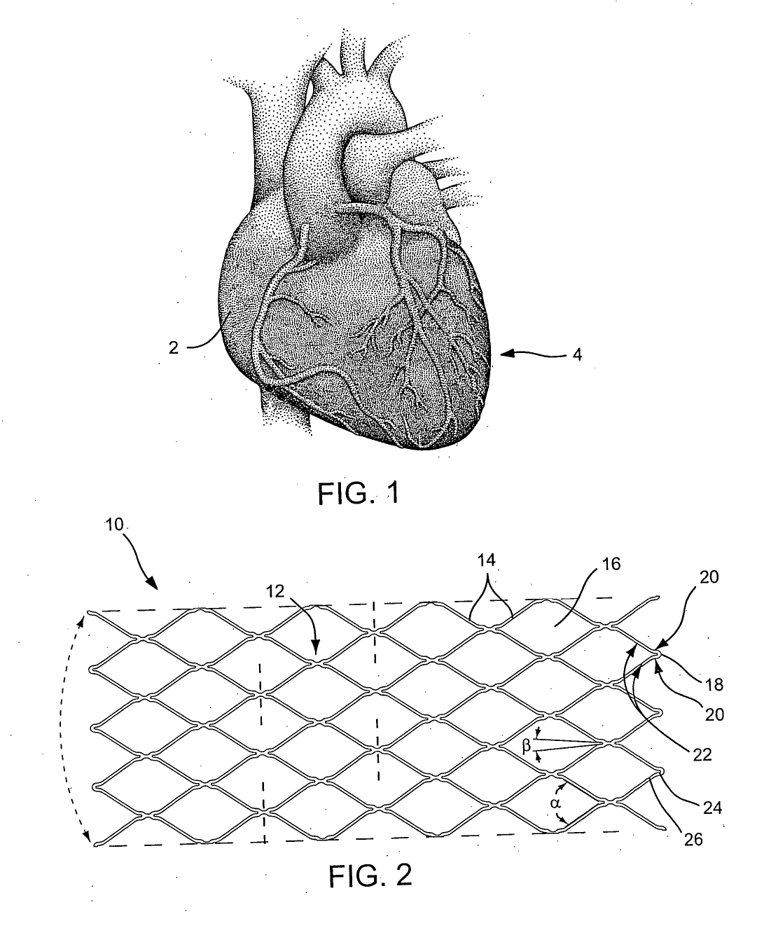

Turning now to FIG. 1, it shows a heart 2 in which its vessels may be the subject of one or more angioplasty and/or stenting procedures. To date, however, significant difficulty or impossibility is confronted in reaching smaller coronary arteries 4. If a stent and a delivery system could be provided for accessing such small vessels and other difficult anatomy, an additional 20 to 25% coronary percutaneous procedures could be performed with such a system. Such a potential offers opportunity for huge gains in human healthcare and a concomitant market opportunity in the realm of roughly $1 billion U.S. dollars—with the further benefit of avoiding loss of income and productivity of those treated.

Features of the present invention are uniquely suited for a system able to reach small vessels (though use of the subject systems s not limited to such a setting.) By “small” coronary vessels, it is meant vessels having a inside diameter between about 1.5 or 2 and about 3 mm in diameter. These vessels include, but are not limited to, the Posterior Descending Artery (PDA), Obtuse Marginal (OM) and small diagonals. Conditions such as diffuse stenosis and diabetes produce conditions that represent other access and delivery challenges which can be addressed with a delivery system according to the present invention. Other extended treatment areas addressable with the subject systems include vessel bifurcations, chronic total occlusions (CTOs), and prevention procedures (such as in stenting vulnerable plaque).

Assuming a means of delivering one or more appropriately-sized stents, it may be preferred to use a drug eluting stent in such an application to aid in preventing restenosis. However, bare-metal stents may be employed in the present invention. The present invention is advantageously employed with self-expanding stents. However, the teachings herein may be adapted for application in the context of balloon-expandable stents.

In any case, features of the present invention are provided in order to hold an implant (e.g., a stent) to be delivered in an access or deployment configuration, after which, the implant assumes its deployed or expanded configuration. Hold-down features may restrain a stent under compressive forces, whereupon release, the stent “springs” open. Alternatively, the stent (or other implant) may simply be secured to the delivery member, where some other mechanism is used to open the stent (e.g., ceasing a flow of chilled saline, thereby allowing a shape memory devices (e.g., NiTi) to warm in order that a material phase change from martinsite to austenite will cause the stent to open).

While some might argue that the particular role and optimal usage of self expanding stents has yet to be defined, they offer an inherent advantage over balloon expandable stents. The latter type of devices produce “skid mark” trauma (at least when delivered uncovered upon a balloon) and are associated with a higher risk of end dissection or barotraumas caused at least in part by high balloon pressures and related forces when deforming a balloon-expandable stent for deployment.

Yet, with an appropriate deployment system, self-expanding stents may offer one or more of the following advantages over balloon-expandable models: 1) greater accessibility to distal, tortuous and small vessel anatomy—by virtue of decreasing crossing diameter and increasing compliance relative to a system requiring a deployment balloon, 2) sequentially controlled or “gentle” device deployment, 3) use with low balloon pre-dilatation (if desirable) to reduce barotraumas, 4) strut thickness reduction in some cases reducing the amount of “foreign body” material in a vessel or other body conduit, 5) opportunity to treat neurovasculature—due to smaller crossing diameters and/or gentle delivery options, 6) the ability to easily scale-up a successful treatment system to treat larger vessels or vice versa, 7) a decrease in system complexity, offering potential advantages both in terms of reliability and system cost, 8) reducing intimal hyperplasia, and 9) conforming to tapering anatomy—without imparting complimentary geometry to the stent (though this option exists as well).

At least some of these noted advantages may be realized using a stent 10 as shown in FIG. 2 in connection with the subject deployment system described in further detail below. Naturally, other stent configurations might be used instead. However, the one pictured is well suited for use in small vessels. It may be collapsed to an outer diameter of about 0.018 inch (0.46 mm), or even smaller to about 0.014 inch (0.36 mm)—including the restraint/joint used—and expand to a size (fully unrestrained) between about 1.5 mm (0.059 inch) or 2 mm (0.079 inch) or 3 mm (0.12 inch) and about 3.5 mm (0.14 inch).

In use, the stent will be sized so that it is not fully expanded when fully deployed against the wall of a vessel in order that it will provide a measure of radial force thereto. The force will secure the stent and offer potential benefits in reducing intimal hyperplasia and vessel collapse or even pinning dissected tissue in apposition.

The stent employed in connection with the subject delivery system preferably comprises NiTi that is superelastic at room temperature and above. Also, it is preferably electropolished. The stent may be a drug eluting stent (DES). Such drug can be directly applied to the stent surface(s), or introduced into an appropriate matrix.

In a 0.014 inch delivery system (one in which the maximum nominal outer diameter of the stent/coating and guide member/restraint have a diameter that does not exceed 0.014 inch), the thickness of the NiTi is about 0.0025 inch (0.64 mm) for a stent adapted to expand to 3.5 mm. Such a stent is designed for use in a 3 mm vessel or other body conduit, thereby providing the desired radial force in, the manner noted above. Further information regarding radial force parameters in coronary stents may be noted in the article, “Radial Force of Coronary Stents: A Comparative Analysis,” Catheterization and Cardiovascular Interventions 46: 380-391 (1999), incorporated by reference herein in its entirety.

As for the stent that may be employed, an optional expanded stent cut pattern 10 is shown in FIG. 2. In one manner of production, the stent is laser (or Electrical Discharge Machining, i.e., EDM) cut from round NiTi tubing, with the flattened-out pattern shown wrapping around the tube as indicated by dashed lines. In such a procedure, the stent is preferably cut in its fully-expanded shape. By initially producing the stent to full size, the approach allows cutting finer details in comparison to simply cutting a smaller tube with slits and then heat-expanding/annealing it into its final (working) diameter. Avoiding post-cutting heat forming also reduces production cost.

Regarding the finer details of the subject stent, necked down bridge or junction sections 12 are provided between adjacent struts 14, wherein the struts define a lattice of closed cells 16. The ends 18 of the cells are preferably rounded-off so as to be atraumatic. To increase stent conformability to tortuous anatomy, the bridge sections can be strategically separated or opened as indicated by broken line. To facilitate such tuning of the stent, the bridge sections are sufficiently long so that fully rounded ends 18 may be formed internally to the lattice just as shown on the outside of the stent if the connection(s) is/are severed to separate adjacent cells 16.

The advantage of the double-concave profile of each strut bridge or junction section 12 is that it reduces material width (relative to what would otherwise be presented by a parallel side profile) to improve trackability and conformability of the stent within the subject anatomy while still maintaining the option for separating/breaking the cells apart.

Further optional features of stent 10 are employed in the cell end regions 18 of the design. Specifically, strut ends 20 increase in width relative to medial strut portions 22. Such a configuration results in a majority of bending (during collapse of the stent) occurring along the length of the struts rather than at the corners of the cells. Longer struts to allow for lower stresses within the stent (and, hence, possibility for higher compression ratios). Shorter struts allow for greater radial force (and concomitant resistance to a radially applied load) upon deployment.

In order to provide a stent that collapses as much as possible (to solid or near-solid structure, such as shown in the fully-loaded systems of the figures) accommodation is made for the stiffer strut ends 20 provided in the design shown in FIG. 2. Namely, the gap 24 between the strut ends 22 is set at a smaller angle as if the stent were already partially collapsed in that area. Thus, the smaller amount of angular deflection that occurs at ends 20 will bring the sections parallel (or nearly so) when the strut medial portions 22 are so-arranged. Radiused sections 26 provide a transition from a medial strut angle a (ranging from about 85 degrees to about 60 degrees) to an end strut angle β (ranging from about 30 to about 0 degrees). In addition, it is noted that gap 24 and angle β may actually be configured to completely close prior to fully collapsing angle α. The value of doing so would be to limit the strains (and hence, stresses) at the strut ends 22 and cell end regions 18 by providing a physical stop to prevent further strain.

By utilizing a design that minimizes strain, very high compression ratios of the stent may be achieved. Compression ratios (from a fully expanded outside diameter to compressed outside diameter—expressed in those terms used by physicians) of as much as 3.5 mm: 0.014 inch (about 10×) are possible—with or without a drug coating and/or restraint used. Compression ratios of 3.0 mm: 0.014 inch (about 8.5×), 3.5 mm: 0.018 inch (about 7.5×), 3.0 mm: 0.018 inch (about 6.5×), 2.5 mm: 0.014 inch (about 7×), 2.5 mm: 0.018 inch (about 5.5×), 2.0 mm: 0.014 inch (about 5.5×), 2.0 mm: 0.018 inch (about 4.5×) offer utility not heretofore possible with existing systems as well.

These selected sizings (and expansion ratios) correspond to treating 1.5 to 3.0 mm vessels by way of delivery systems adapted to pass through existing balloon catheter and microcatheter guidewire lumen. In other words, the 0.014 inch and 0.018 inch systems are designed to corresponding common guidewire sizes. The system may also be scaled to other common guidewire sizes (e.g., 0.22 inch/0.56 mm or 0.025 inch/0.64 mm) while offering advantages over known systems.

While designing the delivery systems to have a crossing profile corresponding to common guidewire sizes, especially for full-custom systems, intermediate sizes may be employed. Still further, it is contemplated that the system sizing may be set to correspond to French (FR) sizing. In that case, system sizes contemplated range at least from 1 to 1.5 FR, whereas the smallest know balloon-expandable stent delivery systems are in the size range of about 3 to about 4 FR.

At least when produced at the smallest sizes (whether in a even/standard guidewire or FR size, or otherwise), the system enables a substantially new mode of stent deployment in which delivery is achieved through an angioplasty balloon catheter or small microcatheter lumen. Further discussion and details of “through the lumen” delivery is presented in the above-referenced “Balloon Catheter Lumen Based Stent Delivery Systems” patent application.

In “small vessel” cases or applications (where the vessel to be treated has a diameter up to about 3.0 mm), it may also be advantageous to employ a stent delivery system sized at between about 0.022 to about 0.025 inch in diameter. Such a system can be used with catheters compatible with 0.022 inch diameter guidewires.

While such a system may not be suitable for reaching the very smallest vessels, in reaching the larger of the small vessels (i.e., those having a diameter of about 2.5 mm or larger), even this variation of the invention is quite advantageous in comparison to known systems. By way of comparison, the smallest known over-the-guidewire delivery system (the “Pixel” system—produced by Guidant) that is adapted to treat vessels between 2 and 2.5 mm has a crossing profile of 0.036 inch (0.91 mm). A system described in U.S. Patent Publication No. 2002/0147491 for treating small vessels is purported to be capable of being made as small as 0.026 inch (0.66 mm) in diameter.

With respect to the Pixel and '491 systems, however, it must be appreciated that a further decrease in stent size may be practically impossible in view of materials limitations and functional parameters of the stent. Instead, the present invention offers a different paradigm for delivery devices and stents that are scalable to the sizes noted herein.

By virtue of the approaches taught herein, it is feasible to design system diameters to match (or at least nearly match) common guidewire size diameters (i.e., 0.014, 0.018 and 0.022 inch) for small vessel delivery applications. As noted above, doing so facilitates use with compatible catheters and opens the possibility for methodology employing the same as elaborated upon below and in the above-referenced “Balloon Catheter Lumen Based Stent Delivery Systems” patent application.

Of further note, it may be desired to design a variation of the subject system for use in deploying stents in larger, peripheral vessels, bilary ducts or other hollow body organs. Such applications involve a stent being emplaced in a region having a diameter from about 3.5 to about 13 mm (0.5 inch). In this regard, the scalability of the present system, again, allows for creating a system adapted for such use that is designed around a common wire size. Namely, a 0.035 to 0.039 inch (3 FR) diameter crossing profile system is advantageously provided in which the stent expands (unconstrained) to a size between about roughly 0.5 mm and about 1.0 mm greater than the vessel or hollow body organ to be treated. Sufficient stent expansion is easily achieved with the exemplary stent pattern shown in FIG. 2.

Again, as a matter of comparison, the smallest delivery systems know to applicants for stent delivery in treating such larger-diameter vessels or biliary ducts is a 6 FR system (nominal 0.084 inch outer diameter), which is suited for use in an 8 FR guiding catheter. Thus, even in the larger sizes, the present invention affords opportunities not heretofore possible in achieving delivery systems in the size range of a commonly used guidewire, with the concomitant advantages discussed herein.

Several known stent delivery systems are compatible with (i.e., may be delivered over) common-sized guides wires ranging from 0.014 inch to 0.035 inch (0.89 mm). Yet, none of the delivery systems are themselves known to be so-sized.

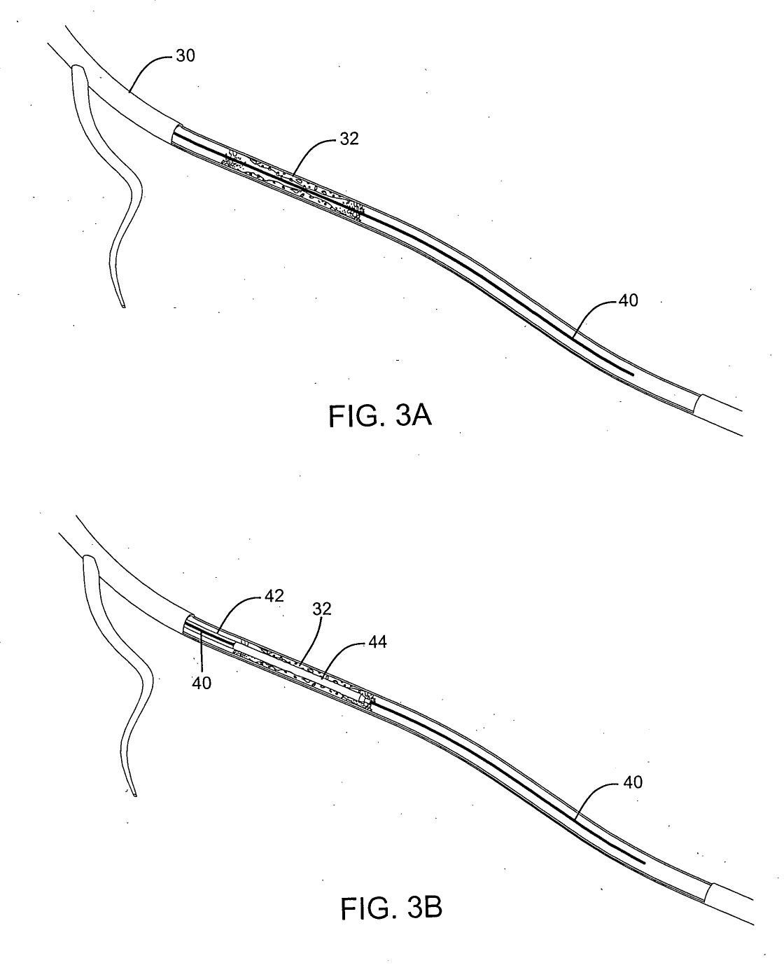

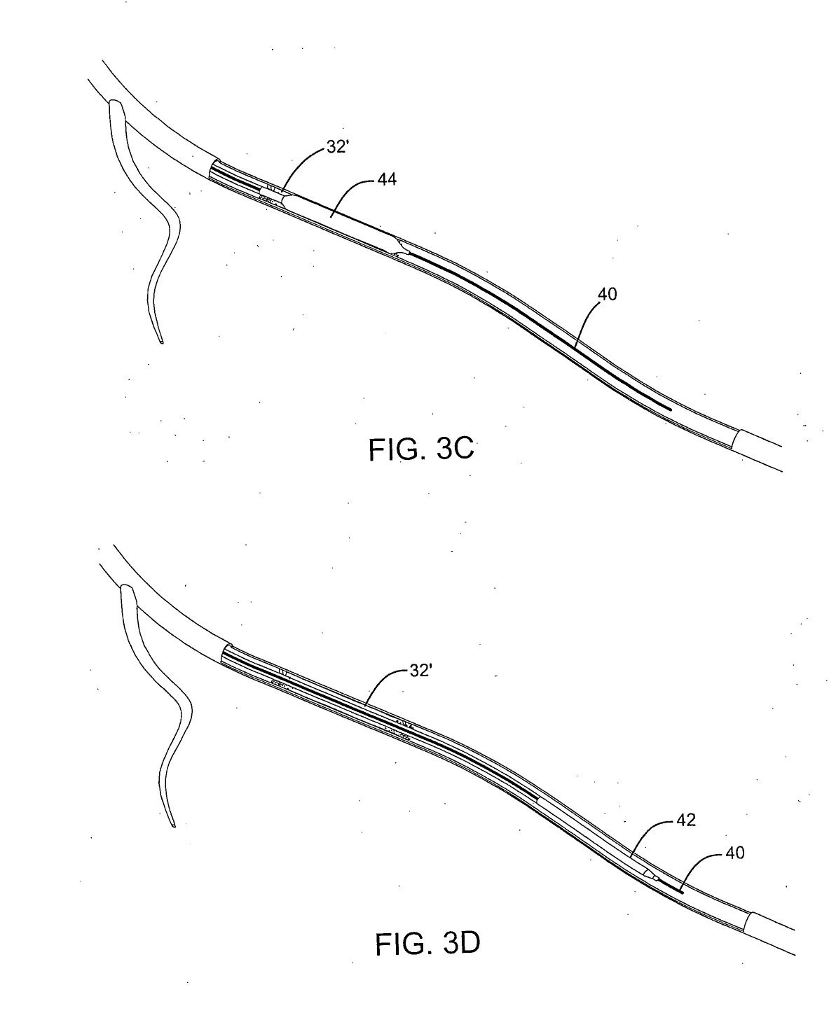

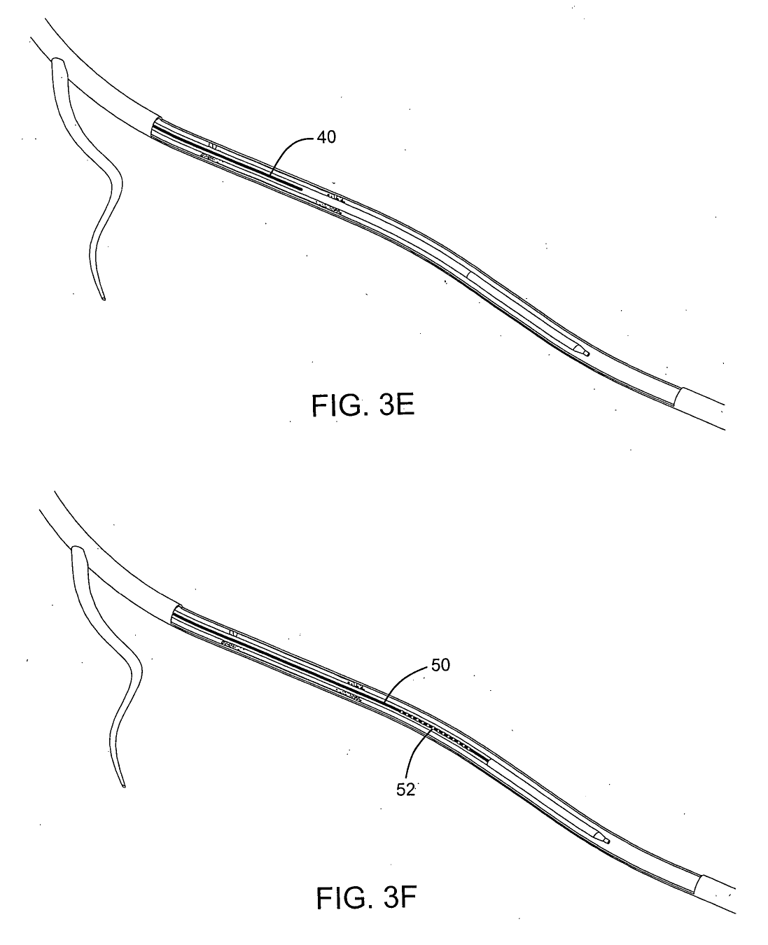

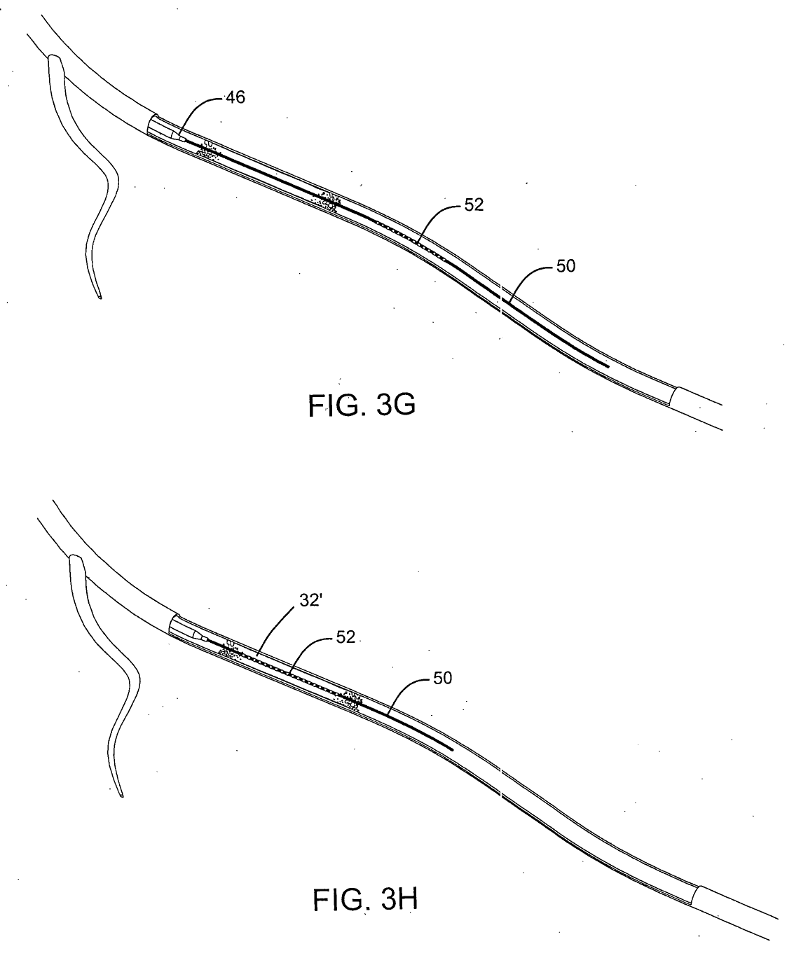

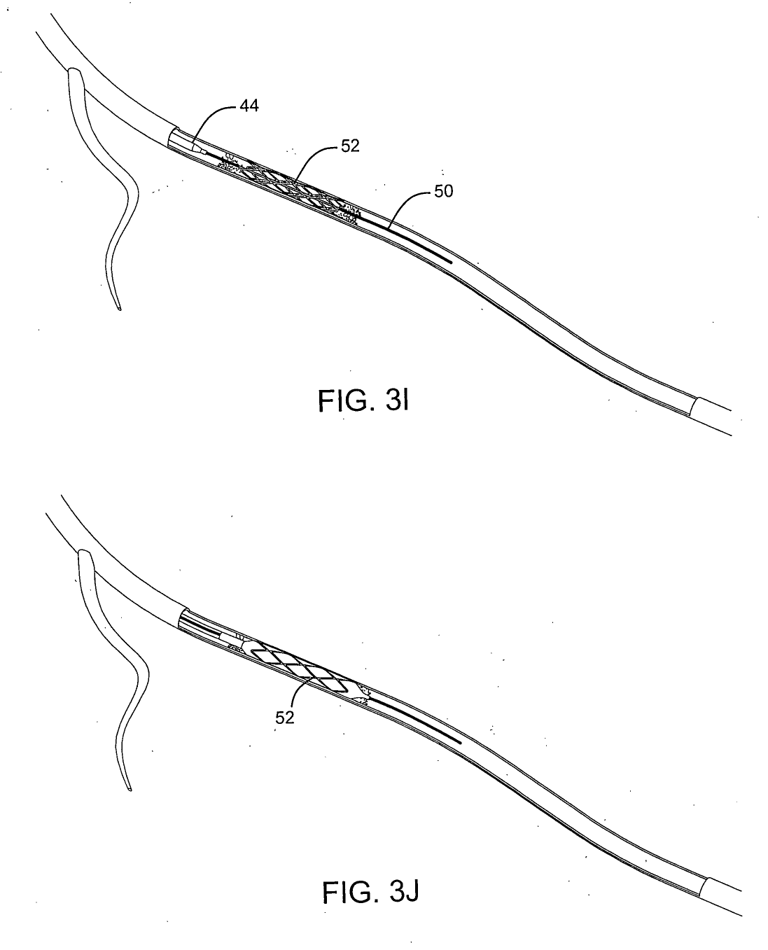

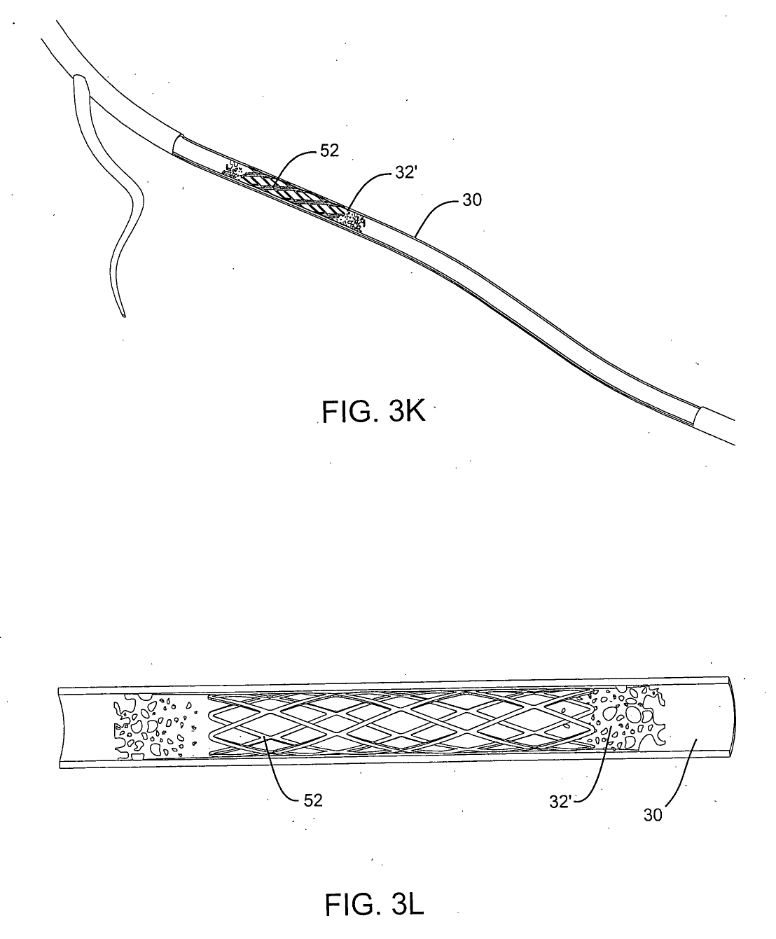

As for the manner of using the inventive system as optionally configured, FIGS. 3A-3L illustrate an exemplary angioplasty procedure. Still, the delivery systems and stents or implants described herein may be used otherwise—especially as specifically referenced herein.

Turning to FIG. 3A, it shows a coronary artery 30 that is partially or totally occluded by plaque at a treatment site/lesion 32. Into this vessel, a guidewire 40 is passed distal to the treatment site. In FIG. 3B, a balloon catheter 42 with a balloon tip 44 is passed over the guidewire, aligning the balloon portion with the lesion (the balloon catheter shaft proximal to the balloon is shown in cross section with guidewire 40 therein).

As illustrated in FIG. 3C, balloon 44 is expanded (dilatated or dialated) in performing an angioplasty procedure, opening the vessel in the region of lesion 32. The balloon expansion may be regarded as “predilatation” in the sense that it will be followed by stent placement (and optionally) a “postdilataton” balloon expansion procedure.

Next, the balloon is at least partially deflated and passed forward, beyond the dilate segment 32′ as shown in FIG. 3D. At this point, guidewire 40 is removed as illustrated in FIG. 3E. It is exchanged for a delivery guide member 50 carrying stent 52 as further described below. This exchange is illustrated in FIGS. 3E and 3F.

However, it should be appreciated that such an exchange need not occur. Rather, the original guidewire device inside the balloon catheter (or any other catheter used) may be that of item 50, instead of the standard guidewire 40 shown in FIG. 3A. Thus, the steps depicted in FIGS. 3E and 3F (hence, the figures also) may be omitted. In addition, there maybe no use in performing the step in FIG. 3D of advancing the balloon catheter past the lesion, since such placement is merely for the purpose of avoiding disturbing the site of the lesion by moving a guidewire past the same.

FIG. 3G illustrates the next act in either case. Particularly, the balloon catheter is withdrawn so that its distal end 46 clears the lesion. Preferably, delivery guide 50 is held stationary, in a stable position. After the balloon is pulled back, so is delivery device 50, positioning stent 52 where desired. Note, however, that simultaneous retraction may be undertaken, combining the acts depicted in FIGS. 3G and 3H. Whatever the case, it should also be appreciated that the coordinated movement will typically be achieved by virtue of skilled manipulation by a doctor viewing one or more radiopaque features associated with the stent or delivery system under medical imaging.

Once placement of the stent across from dilated segment 32′ is accomplished, stent deployment commences. The manner of deployment is elaborated upon below. Upon deployment, stent 52 assumes an at least partially expanded shape in apposition to the compressed plaque as shown in FIG. 31. Next, the aforementioned postdilatation may be effected as shown in FIG. 3J by positioning balloon 44 within stent 52 and expanding both. This procedure may further expand the stent, pushing it into adjacent plaque—helping to secure each.

Naturally, the balloon need not be reintroduced for postdilatation, but it may be preferred. Regardless, once the delivery device 50 and balloon catheter 42 are withdrawn as in FIG. 3K, the angioplasty and stenting procedure at the lesion in vessel 30 is complete. FIG. 3L shows a detailed view of the emplaced stent and the desired resultant product in the form of a supported, open vessel.

In the above description, a 300 cm extendable delivery system is envisioned. Alternatively, the system can be 190 cm to accommodate a rabid exchange of monorail type of balloon catheter as is commonly known in the art. Of course, other approaches may be employed as well.

Furthermore, other endpoints may be desired such as implanting an anchoring stent in a hollow tubular body organ, closing off an aneurysm, delivering a plurality of stents, etc. In performing any of a variety of these or other procedures, suitable modification will be made in the subject methodology. The procedure shown is depicted merely because it illustrates a preferred mode of practicing the subject invention, despite its potential for broader applicability.

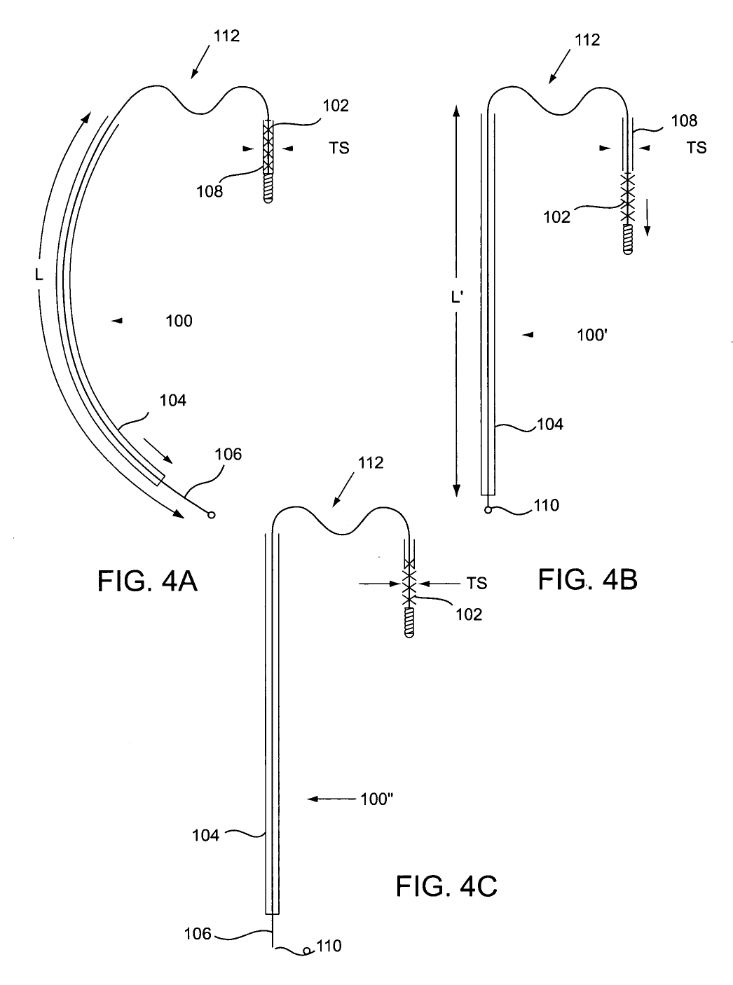

As for the potential aforementioned problems with various stent delivery systems, FIGS. 4A-4C illustrate those associated with simple sheath based self-expanding stent delivery. FIGS. 4A and 4B explain observed forward-wandering stent deployment behavior in a simple-sheath based delivery system whereas FIG. 4C illustrates the proper mode of deploying a stent with such a system.

As noted above, careful actuation of a sheath-based delivery system will not necessarily ensure precise stent placement. If one does not draw the slack out of the system then the stent and any associated tip (if one is provided—such as in the device described in U.S. Patent Application Attorney Docket No. CRMD-008 filed on even date herewith), can be moved forward upon withdrawal of the device stent restraining sheath.

Specifically, FIG. 4A shows a delivery system 100 in a first state. In this state, it is shown curved to indicate the slack in the system. Of course, while in use the system is not so-shaped. Rather it approximately follows the anatomy and/or interior of guide catheters, microcatheter, etc. employed to allow the device to navigate so the stent 102 the device carries is located at the target site (“TS”).

Theoretically, then, if one were to withdraw the proximal sheath end 104 back over the device's core wire 106, it would be expected that the distal sheath end 108 would follow, thus releasing the stent at the target site.

However, this will not likely be the case. Instead, the stent will move forward as shown in FIG. 4B. The reason is that the delivery device assumes a second state 100′ in which pulling sheath proximal end 104 toward the core member end 110 simply takes the curvature out of the system, drawing it relatively taught in the subject anatomy.

This action will occur since (relatively speaking) the delivery device body is stuck or anchored within tortuous region 112 shown in reduced scale. With the distal end of the sheath unable to easily move, the system accommodates the user input by the core member 106 and associated stent pushing forward as the arced path length (“L”) within the sheath is shortened to a straight (or straighter) path line L′. The stiction in the tortuous section results from bending the device around the anatomy and the frictional engagement resulting from the contact force.

Of course, lubricious materials or coatings can be (and are applied) to reduce the effect. However, en balance, given the system flexibility requirements and anatomy to be accessed for stent deployment, the noted action still occurs given a starting point as shown in FIG. 4A.

However, if one is to set the system up as shown in FIG. 4C by first drawing the slack out of the system 100″ in a third state as shown, then proper stent placement at the target site TS can be achieved. When under some overall tension, and with no ability for loosing arc length in the system, pulling proximal sheath end 104 caused distal end 108 to follow, releasing stent 102 as desired.

While such manipulation of the device sounds rudimentary (i.e., drawing the slack out of the system before attempting sheath actuation), the procedure can be quite delicate. If the system is not retracted with enough force, slack will remain and the condition in FIG. 4B will result. If too much tension on the system is applied, then the stent 102 can be inadventerntly drawn proximally of the target site. Negotiating an appropriate balance is no small feat, and requires a very high degree of skill and training.

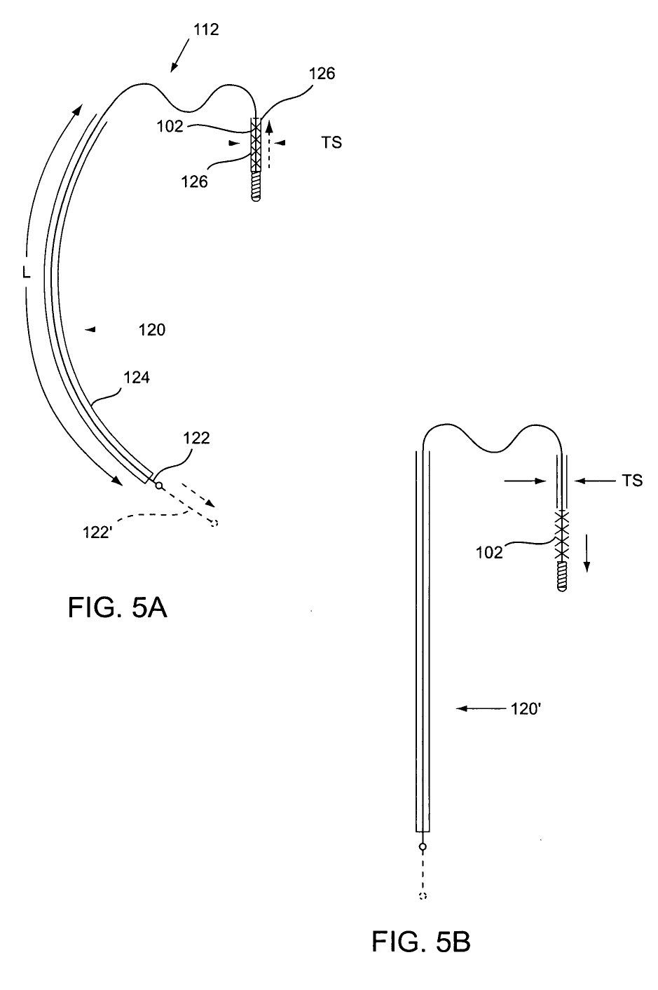

When properly configured (or the correct one of a plurality of configurations is selected for a given task), a device according to the present invention can deliver a stent to a precise target site or location even in the configuration shown in FIG. 5A. In this figure, a delivery guide device 120 employing a moveable inner or core member 122 within an outer hypotube or sleeve 124 is provided, in which the inner member actuates a distal restraint 126 that holds a stent 102 in a collapsed configuration until the restraint is pulled from the stent.

By “properly configured” it means that the restraint is in fact able to slide off of the stent, rather than being bound-up by or in (or within a catheter lumen) tortuous region 112. With a system that is not correctly selected for the subject anatomy (or when a Radius™ system as referenced above is employed), the condition shown in FIG. 5B may result. In that case, the delivery device 120′ in the second state is required to straighten-out and push stent 102 forward in much the same manner as with the simple sheath system.

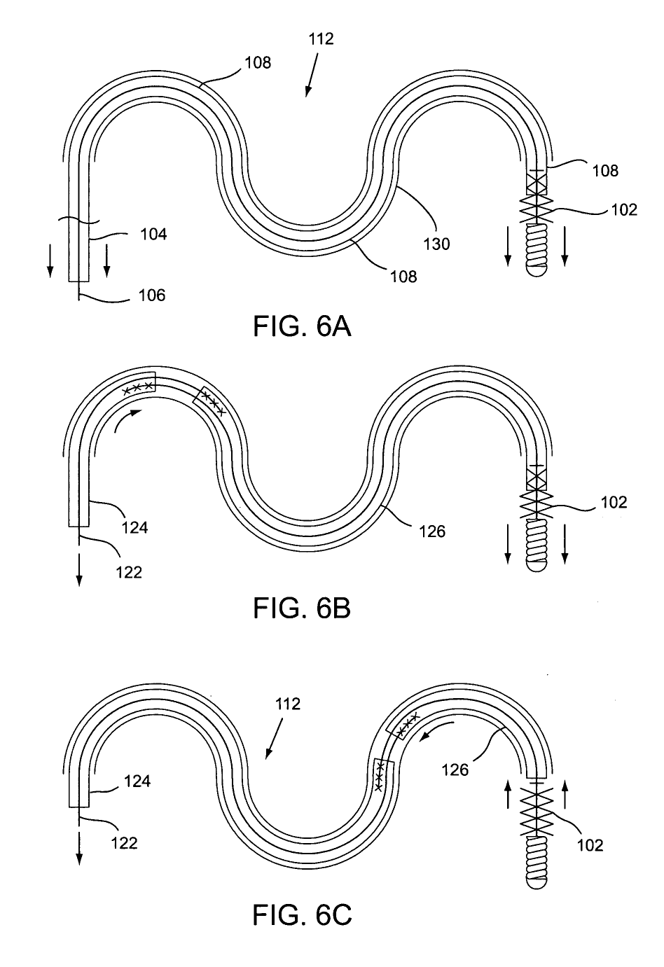

In order to illustrate what is occurring in the tortuous section/region 112, in FIGS. 4A-5B, details thereof are provided in FIGS. 6A-6C (with action depicted by arrows). In FIG. 6A, a simple-sheath system is shown. Here, it is clear as to the manner in which the distal portions 108 of the sheath are prone to suffer from stiction with the vessel walls 130 when disposed about highly curved sections.

Similarly, in a system according to the present invention where the length selected of the restraint 126 is quite long as shown in FIG. 6B, it too may be prone to stiction. However, where the restraint is shorter as in FIG. 6C, sleeve 124 will tend to anchor the delivery device within the body conduit against walls 130, and restraint 126 will slide freely.

As provided in the Summary above, the relative lengths of these members will vary. Such variation and tuning of lengths within the invention will also be in some cases anatomy or application specific.

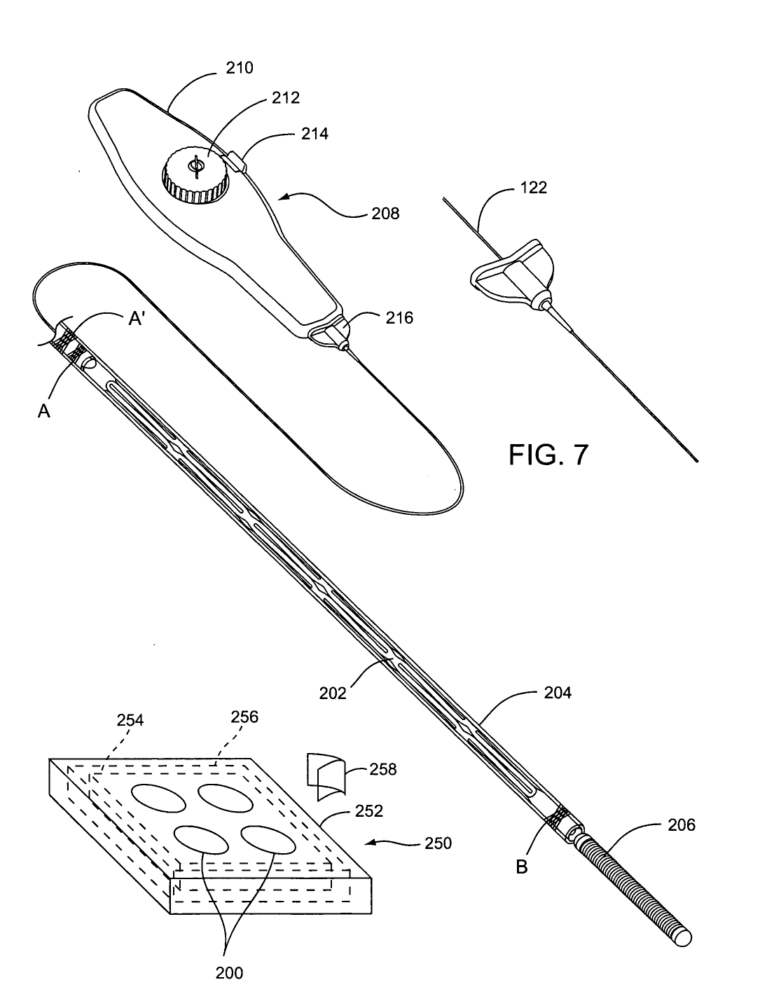

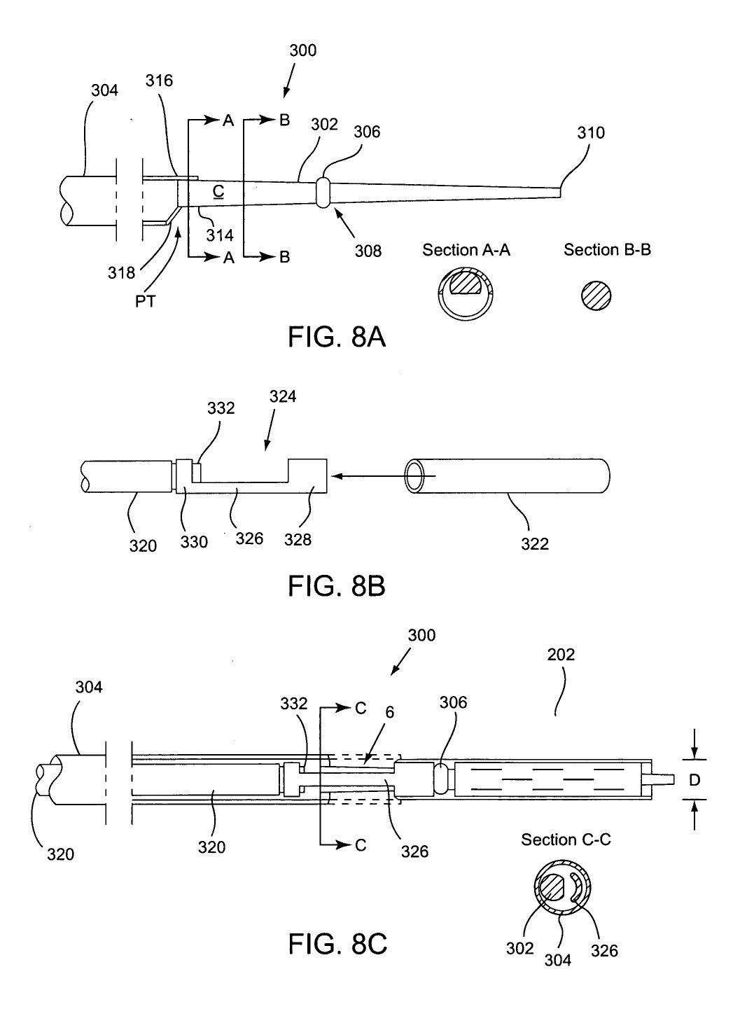

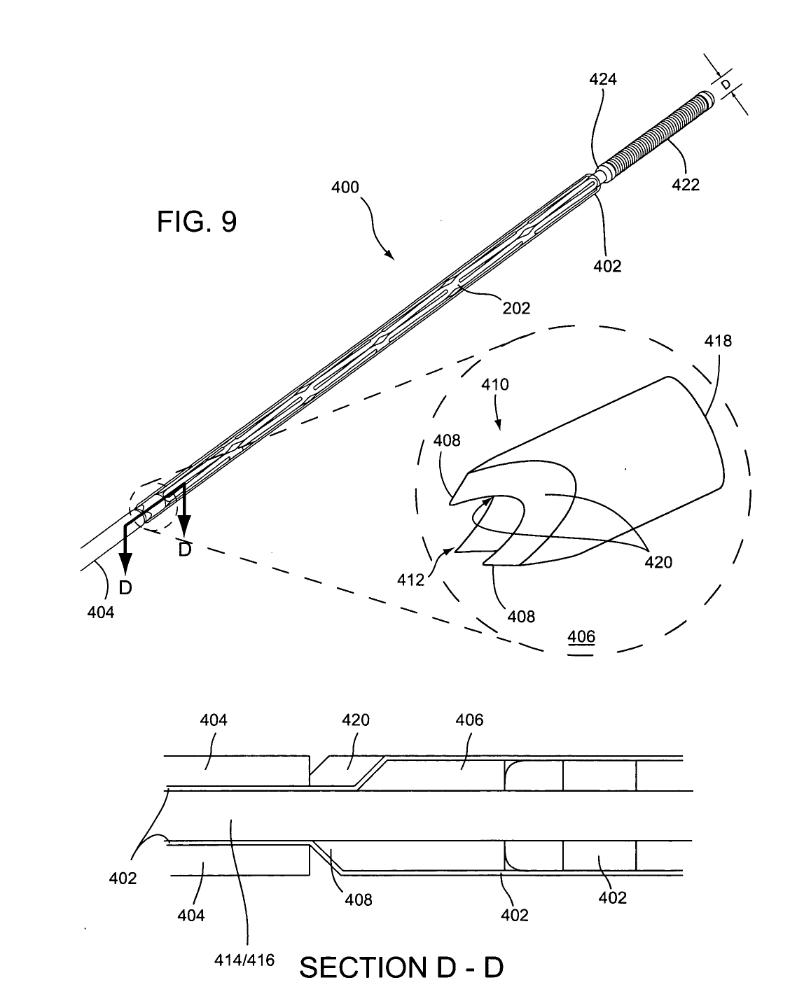

As for an overview of the subject delivery systems, FIG. 7 is provided. In FIG. 7, a delivery system 200 is shown along with a stent 202 held thereon in a collapsed configuration. A restraint 204 is provided over and around the stent. The restraint may fully surround the stent or only subtend a partial circumference of the stent, it may be split, splittable, comprise a plurality of members or be otherwise provided around the stent to hold or restrain it in a collapsed profile. Additional optional details are discussed in connection with FIGS. 8A-9 below. The delivery device or system preferably comprises an atraumatic distal tip 206 of one variety or another. On the other end of the system, a custom handle 208 is preferably provided.

The handle shown is adapted for rotable actuation by holding body 210, and turning wheel 212. It may include a lock 214. Furthermore, a removable interface member 216 facilitates taking the handle off of the delivery system proximal end 218. The interface will be lockable with respect to the body and preferably includes internal features for disengaging the handle from the delivery guide. Once accomplished, it will be possible to attach or “doc” a secondary length of wire 220 on the delivery system proximal end, allowing the combination to serve as an “exchange length” guidewire, thereby facilitating changing-out the balloon catheter or performing another procedure. Alternatively, the wire may be an exchange-length wire.

FIG. 7 also shows packaging 250 containing any of a number of coiled-up delivery systems 200. When a plurality are provided, they are typically configured in support of a methodology where an appropriate one is picked to reach a target site and deploy a stent without unintended axial movement of the same (whether or not appreciable slack is drawn out of the system prior to deployment).

The packaging may serve the purpose of providing a kit or panel of differently configured delivery devices. In the alternative, the packaging may be configured as a tray kit for a single delivery system. Either way, packaging may include one or more of an outer box 252 and one or more inner trays 254, 256 with peel-away coverings as is customary in packaging of disposable products provided for operating room use. Naturally, instructions for use can be provided therein. Such instructions may be printed product or provided in connection with another readable (including computer-readable) medium. The instructions may include provision for basic operation of the subject devices and/or the selection methodology.

Regarding the specifics of the distal restraint employed in the delivery guides, it preferably is one that does not have a section that increases in size during, or after, deployment of the stent. Two classes of exemplary restraint devices are shown, first, in FIGS. 8A-8C, then in FIG. 9. They are such that the restraint diameter remains constant or actually decreases in diameter upon withdrawal from the stent and release of the same.

Regarding the first variation shown in FIGS. 8A-8C, FIGS. 8A and 8B show sub-assemblies, whereas the parts are combined in FIG. 8C. In FIG. 8A, a distal end 300 of the delivery device 200 is show in partial cross-section. It includes an extension wire 302 attached/connected to a hypotube section 304. The hypotube extends proximally (to the left) and serves as the “sleeve” referred to above. For a “one-sided” system as further detailed in FIGS. 8B and 8C, the extension member 302 is offset within the tubing 304. This relation of elements is most clearly shown in Section A-A, showing a pass-through opening (“PT”) between the extension 302 and sleeve 304.

Distal of the connection, a shoulder section may be ground into the wire or a separate ring 306 may be attached thereto to provide a stop surface 308 for abutting the stent to be delivered. Moving toward the distal tip 310, the system may be tapered as shown. The length of the extension over which the restraint rides is variable as is the restraint. The taper may be desired for increased distal flexibility.

The overall length of the system from a distal tip (possibly incorporating an atraumatic tip, to the base of any actuation device provided may be around 135 cm (53 inch) to 200 cm (79 inch) or more preferably between 180 cm (71 inch) and 190 cm (75 inch). Overall longer or shorter system lengths are also contemplated. The length of the extension 302 and stent-overlying restraint/connector is variable. The length of the extension section may be between about 10 cm to 15 cm, about 15 cm to about 25 cm or up to 30 cm or longer as possibly influenced or dictated by-system flexibility requirements. Yet, in support of the subject “anchoring” methodology, the exposed length of the restraint and/or connector bridge sections will be as characterized in the Summary above (either in explicitly, or implicitly by virtue of noted functional parameters).

Regarding such other specifics of the delivery guide,- it may be desired to create a flat section 312 for clearance purposes where the proximal end 314 of the extension member and distal end 316 of the sleeve overlap. To increase system compliance at this intersection, it may be desirable to relieve or create an angled section 318 at the distal end of the hypotubing. To encourage even navigation performance characteristics, extension wire 302 will return to round as shown in Section B-B distal of the intersection.

FIG. 8B shows the remaining elements for the distal portion 300 of this variation of the delivery system. Specifically, an inner wire 320, a restraint 322 and connector 324 for attachment to each piece is shown. Because the connector (in essence) crosses from the outside diameter “D” of the device to the inside, it includes a bridge section 326 to traverse pass-through PT. Bonding sections 328, 330 are provided, preferably for gluing to the restraint and a distal end 332 of the inner wire 320.

Of course, the restraint and the bridge section may be provided integrally. Otherwise, they may be made of different materials. For example, the connector (which includes the bridge section) may comprise stainless steel or Nitinol (just as other members of the delivery guide) and the restraint may comprise a polymeric material. A polymeric restraint (such as polyamide or PET may be desired since it is readily obtained in very thin walled tubing—down to 0.00025 inch).

In FIG. 8C, the various components discussed above are assembled to form a complete delivery guide, with the distal portion shown in partial cross-section as in FIG. 8A. Section C-C shows the manner in which bridge section 326 passes by extension section 302 within the device. Further, a stent 202 is shown in a collapsed configuration within the restraint, thereby completing the delivery system.

Overall, the system has a diameter (“D”) dictating its crossing profile. To actuate the delivery guide device, inner/core member 320 is withdrawn causing the restraint to slide off of the stent without the diameter D to increase as the clearance gap (“G”) between the restraint and sleeve is closed.

Note, however, that it may be the case that no open gap G is provided. This may be accomplished by extending the sleeve over the connector as indicated by dashed/phantom lines in FIG. 8C over the gap. Indeed, this “hidden” bridge or connector variation of the invention may be desirable in order to help prevent system kinking, pushing the restraint forward after retraction (such as is in an abortive stent delivery procedure) or just to generally protect any bridge section(s). The sleeve may be extended by a greater length of hypotubing or an extension sleeve or tube thereto (e.g., made of a thinner polymeric material). Further details of the same are presented in U.S. Patent Application Atty Docket No.: CRMD-005 entitled “Corewire Actuated Delivery Systems with Fixed Distal Stent-Carrying Extension” referenced above.

As to the restraint variation of the invention in FIG. 9, it operates in a substantially different manner. Still the delivery device 400 diameter D does not increase during or after stent 202 release.

In fact, the system operates by employing a diameter-reducing restraint 402. In short, the restraint (itself) is pulled back into sleeve 404 as opposed to merely a bridging portion connecting the restraint to the inner member of the system. Such action may be facilitated by using a pre-split restraint or restraint having a plurality of sections. Alternatively, the restraint may be separated (e.g. along a perforation line or lines). This may be facilitated by a wedge type member. Still further, the restraint may be cut into sections by one or more opposing blade members.

The delivery guide in FIG. 9 is configured for such operation. In this case, a stent stop or interface member 406 is provided. In the enlarged detail of the same, one can clearly see blade portions 408. In this example, the blades are formed by cutting tubing on a bias at a proximal end 410. The lumen 412 defined by the tubing accepts either the inner/core member running the full length of the device 414 or an extension wire 416 like that shown in FIG. 8A and 8C. A distal end 418 of the interface member provides a proximal stop section for the stent.

As to the specific manner of operation, Section D-D is provided to help explain. In this sectional view, restraint 402 is shown diving down from outside of the stent 202 to within the sleeve 404. The sections of the restraint that are cut (or separated) pass through recesses 420.

In some variations of the invention, the inner member that is actuated to withdraw the restraint may be an extension of the restraint itself, a tubular member connected thereto that runs the length of the system or it may be a core member 414. If it is a core member 414, then (as stated above) it may be desirable to include an extension wire 416, so that the stent is disposed over such a member and an atraumatic tip 422 can be provided at a distal end. In which case, a proximal portion of the distal tip may provide a distal stop surface abutting the stent.

In any case, further constructional details of the diameter-adaptive restraint may be appreciate in reference to the incorporated patent application describing the same; likewise for the sliding restraint system shown in FIGS. 8A-8C.

In regard to any such system, it is to be understood that conventional materials and techniques may be employed in the system construction (in any of the packaging, handle, delivery guide and/or stent). In this regard, it will often be desired to provide a lubricious coating or cover between moving components to reduce internal system friction. As for other options of controlling lubriciousness (or intentionally providing a lack thereof) reference is made to the Summary of the invention above.

In addition, it is to be understood that various radiopaque markers or features may be employed in the system to 1) locate stent position and length, 2) indicated device actuation and stent delivery and/or 3) locate the distal end of the delivery guide. As such, various platinum (or other radiopaque material) bands or other markers (such as tantalum plugs) may be variously incorporated into the system. Alternatively, or additionally, the stent stop or blocker member may be made of radiopaque material. Especially where the stent employed may shorten somewhat upon deployment, it may also be desired to align . radiopaque features with the expected location (relative to the body of the guide member) of the stent upon deployment. For example, it may be desired to incorporate radiopaque features into the restraint and/or bridge or connector sections so that the deployment motion of the device is visible under fluoroscopy. Exemplary markers that may be of use are shown at a proximal end of the stent in FIG. 7 as elements A and A′—on the delivery guide body and restraint, respectively - and at a distal end of the stent on the restraint as element B.

Though the invention has been described in reference to several examples, optionally incorporating various features, the invention is not to be limited to that which is described or indicated as contemplated with respect to each embodiment or variation of the invention. The breadth of the present invention is to be limited only by the literal or equitable scope of the following claims. That being said, we claim:

1. A stenting method comprising:
selecting a stent delivery system from a plurality of such systems,
wherein each of the systems comprises a self-expanding stent and a delivery guide, the delivery guide comprising an inner member, an outer sleeve, a stent stop fixed in position relative to the outer sleeve and a distal restraint having a length slidingly disposed over the stent to hold the stent in a collapsed configuration until withdrawal of the restraint to deploy the stent,
locating the delivery system so the stent is at a treatment site, and
actuating the inner member to withdraw the restraint to release the stent for deployment at the treatment site without axial movement of the stop,
wherein a non-selected system would produce axial movement of the stop during stent deployment.

2. The method of claim 1, wherein the delivery system does not increase in diameter after releasing the stent.

3. The method of claim 1, wherein the treatment site is a lesion in a blood vessel or graft.

4. The method of claim 3, wherein the blood vessel or graft is in the coronary vasculature.

5. The method of claim 4, wherein the selected delivery guide has an exposed length of restraint that between about a length of the stent and about 5 cm long.

6. The method of claim 5, wherein the selected delivery guide has an exposed length of restraint that between about 5 cm and about 10 long.

7. The method of claim 5, wherein the selected delivery guide has an exposed length of restraint that between about 10 cm and about 20 cm long.

8. The method of claim 4, wherein in locating the stent, a distal end of the sleeve or an extension therefrom is located up to about 30 cm distal of the ostium.

9. The method of claim 8, wherein in locating the stent, a distal end of the sleeve or an extension therefrom is located up to about 20 cm distal of the ostium.

10. The method of claim 6, wherein in locating the stent, a distal end of the sleeve or an extension therefrom is located up to about 10 cm distal of the ostium.

11. The method of claim 1, wherein the selecting is between only two stent delivery systems.

12. The method of claim 1, further comprising providing the plurality of stent delivery systems.

13. The method of claim 1, further comprising advancing the delivery system through a catheter lumen having a proximal hemostatic valve, and closing the valve onto the sleeve prior to the withdrawing of the inner member.

14. The method of claim 1, wherein the treatment site is within neurovasculature.

15. The method of claim 1, wherein the treatment site is within an organ selected from the kidney and liver.

16. The method of claim 1, wherein the treatment site is within reproductive anatomy.

17. The method of claim 16, wherein the reproductive anatomy is selected from vasculature, vasdeferens and fallopian tubes.

18. The method of claim 1, wherein the treatment site is distal to tortuous anatomy, the tortuous anatomy having a diameter of less than about 3 mm over a length of at least about 5 cm

19. The method of claim 18, wherein the diameter is less than about 2.5 mm.

20. The method of claim 19, wherein the diameter is less than about 2.0 mm.

21. The method of claim 18, wherein the length is at least 10 cm.

22. The method of claim 21, wherein the length is at least 15 cm.

23. A stenting system comprising:
a self-expanding stent;
a delivery guide member comprising an inner member, an outer sleeve, a stent stop fixed in position relative to the outer sleeve, and a distal restraint having a length slidingly disposed over the stent to hold the stent in a collapsed configuration until release,
wherein withdrawal of the inner member within the sleeve and withdrawal of the restraint or a connection to the restraint from outside of the sleeve to inside of the sleeve releases the stent, and
wherein the delivery system does not increase in diameter during or after releasing the stent.

24. The system of claim 23, wherein the delivery guide is adapted to release the stent without axial movement of the stop.

25. The system of claim 23, wherein the restraint is has an exposed length, the length adapted to avoid stiction with apposing anatomy.

26. The system of claim 23, wherein the restraint has a lubricious outer coating.

27. The system of claim 23, wherein the sleeve is adapted to grip apposing anatomy.

28. The system of claim 27, wherein the sleeve has no lubricous coating on at least a distal end.

29. The system of claim 23, further comprising a connector member between the restraint and inner member.

30. The system of claim 24, wherein the delivery guide is adapted to release the stent without axial movement of the stop when releasing the stent at a target site within a coronary artery.

31. The system of claim 23, further comprising a removable handle for actuating the inner member.

32. The system of claim 23, further comprising an atraumatic distal tip.

33. The system of claim 32, wherein a proximal end of the atraumatic distal tip abuts the stent.
